(19) United States
(12) Patent Application Publication
(10) Pub. No.: US 2002/0156340 A1
(43) Pub. Date: Oct. 24, 2002

(54) Proprioceptive device that contains nutrients for the treatment of stratural diseases

(76) Inventor: Edward Ludwig Blendermann, Petersburg, TN (US)

Correspondence Address: Edward L. Blendermann, 2758 Lewisburg Hwy, Petersburg, TN, 37144, US

(21) Appl. No.: 10/075,221

(22) Filed: Feb. 13, 2002

Related U.S. Application Data
(60) Provisional application No. 60/267,728, filed on Feb. 12, 2001.

(51) Int. Cl. A61N 2/00

(52) U.S. Cl. 600/15; 128/897

(57) ABSTRACT

An article such as a locket (sole insert, patch, or other device to hold nutrients in on the body), which incorporates at least one nutrient, which is capable of emitting electromagnetic waves that stimulate proprioceptive zones throughout the body, resulting in a therapeutic effect for the treatment and/or the prevention of problems connected to statural equilibrium.

### REFERENCES SITED

| | | |
|---|---|---|
| 6,148,822 | Nov 21, 2000 | CRON, et al. |
| 5,158,526 | Oct 1992 | BRICOT |

### BACKGROUND OF THE INVENTION

A. Field of the Invention

The present invention relates to a medical device and devices that have a therapeutic effect for the treatment and/or the prevention of problems connected to statural equilibrium.

B. Description of the Prior Act

Chronic pains in the back, head, and neck are associated with faulty posture resulting from statural disequalibrium.

Ten percent or less of the population have a perfectly balanced pelvic, pectoral, and head symmetry. These individuals rarely experience back and neck pain and are rarely prone to orthosis. Imbalances are common in the remaining ninety percent of the population who do experience spinal curvatures or non-physiologic intervertebral angles, leading to muscular spasms, articular and bone pain.

Poor posture can also be responsible for various disorders, including (arthosis, lumbago, vertigo, muscular and articular pain, and scoliosis. Observation that correction of the pelvic and pectoral girdle imbalances, and head tilt establishes a link between these disorders and the spine in that such correction results in improvement of symptoms.

Spinal equilibrium restoration has been achieved by various therapeutic approaches that have been developed. The majority of these involve pedal devices in the soles, being that the feet are the points of normal support of the human body. Furthermore, it has been proven that the feet are the proprioceptor neural chain origins that control the tonus of the statural muscular system, of which the spinal, pelvic, and pectoral muscles are components. By adequate stimulation of particular reflex zones located on the soles of the feet, there is a re-alignment of the ascending proprioceptive chains and thus a re-equilibrium of the skeletal structure.

This concept is evident by the design of the following types of soles:

Micro-relief soles located at the level of reflex zones on the feet have been used for several years. However, these types of soles are limited by the fact that the micro-reliefs must be meticulously adjusted for each patient; an adjustment of their thickness must be regularly modified. Furthermore, they are only activated in the standing position.

There are also soles that utilize a polarizing substance, such as a plate that has been described in U.S. Pat. No. 5,158,526. These polarizing plates have been marketed in Europe and have been used for several years for structural disorders. Their clinical use is limited in that they must be applied continuously in direct contact with the soles of the feet. Their efficacy is almost eliminated when fabrics such as cotton or nylon (found in socks) are placed between the device and the skin. The result is a problem in using the device due to poor patient compliance due to the uncomfortable audition created by perspiration and friction. The device must also be monitored for efficacy due to the short lifetime of the active principles being only several months.

A sole of superposed polarizing plates is described in U.S. Pat. No. 5,158,526 (Briscot). Briscot describes a method and apparatus for treating reflex zones of the feet by applying “polarized undulatory energy flux generated by terrestrial fields or radiations to the reflex zones of the feet.” He also teaches that microcrystals may be used in his device applied to the sole. He explains the absolute necessity to process said microcrystals into a polarizer under specific conditions such that crystals be properly oriented. Briscot maintains that prior art only stimulate one or two proprioceptive chains, and therefore by covering the entire sole his device would stimulate other zones which would “modulate” the activation of the proprioceptive chains.

A sole containing a flexible filtering film (Canadian Pat. No. 1,328,734) has been found to similarly affect those reflex zones and correct posture.

From inventors Cron, Patuot and Abribat, U.S. Pat. No. 6,024,093 and U.S. Pat. No. 6,148,822, both describe a proprioceptive sole or pedal device containing crystals for treatment of statural disorders. The advantage of this device is that it does not have a polarizing element that must be monitored; it may be worn under the socks for comfort. They also provide a type of sole with a clinical efficacy proven by a placebo-controlled, double-blind study. The active agent in this sole is a crystalline substance derived from various crystals. Both the type and amount of crystalline substances to be used, in combination or alone, are selected on the ability to stimulate the reflex zones of the feet:

Although the exact mechanism involved in alleviating musculoskeletal pains associated or not with the spine disequilibrium and troubles associated with statural disequilibrium are not understood well, it is believed that these crystalline substances produce natural frequencies and radiations which affect the reflex zones.

The ability of these crystals to therapeutically affect reflex zones on the feet through the delivery of natural frequencies is proven by a double-blind study that was submitted in these patents.

These patents cited all involve a device containing a means of stimulating reflex zones on the feet which trigger a proprioceptive efferent signal to the central nervous system and a resulting efferent signal back to the muscular system which moves bones back into alignment.

Such an increase in muscle tone was observed by George Goodhart D. C. in 1970 when a nutrient inside a subject's mouth was chewed. Goodhart would than observe a phenomenal increase of strength pf weak interior muscles. (Interestingly, the lower jaw and the TMJ are highly innervated by proprioceptive fibers.) Goodhart must be credited with his observation of increasing muscle tone through stimulation caused by nutrients.

In the last thirty years, this ability of nutrients to make week muscles stronger has been embraced by nutritionists, kinesiologists, chiropractors, and physical therapists in testing nutrients. A large proportion of these also utilize the topical placement of various nutrients to result in the clinical observation of weak muscles becoming stronger.

It is the contention of this inventor that the soles of the feet are not solely responsible for the body's enormous proprioceptive input, which allows for multiple placement sites for a reflex zone stimulation.

Therefore, it is an object of this invention to provide stimulation of proprioceptive reflex points throughout the surface of the body by the topical placements of nutrients. Using a variety of nutrient-holding devices the subject is allowed to wear the agents and thus be afforded the envisioned therapeutic effect continually. In addition, several zones of stimulation can be utilized in addition to, separate from, the soles of the feet. The result is to moderate the activation of proprioception.

The advantage of this invention over prior art is that the device may be used on various sites on the body. The active principle may be incorporated into clothing, thereby increasing patients' compliance.

The active principle may be used on other locations on the body surface, thereby avoiding the constant wear and tear associated with any pedal device mentioned in prior art. The active principle may be in the form of a solid, powder, gel or liquid, making it more diversified and adaptable to different types of location and device type.

It is an object of the present invention to provide a means (active principle) being nutrients (metabolites) to allow the function of rapidly correct structural asymmetries (faulty posture) and thus alleviate associated physical disorders such as back and neck pains.

Is it another object of the invention to provide a nutrient-holding device that may be placed on the body in a variety of locations in addition to, and including, being placed on the soles of the feet.

It is another object of the invention to provide a device containing nutrients (metabolites), of which these active agents would be worn under clothing (i.e., socks and stockings) and still remain effective.

It is another object of the invention to provide a nutrient-holding device that may be incorporated into furniture (i.e., mattresses or chairs) and also into quilts or pillows to administer the active principles while resting.

It is another object of the invention to provide a nutrient holding device that has a long lifetime, in order to avoid the need for frequent monitoring of the quality of said device,

It is another object of the invention to provide a nutrient holding device with a clinical efficacy resulting from data received from a placebo-controlled, double blind study.

It is another object of the invention to promote a nutrient holding device where said nutritional supplements may be encapsulated in small stainless steel balls and combined with dental amalgam as dental fillings to stimulate the rich proprioceptive reflex zones at the jaw.

It is another object of the invention to provide a nutrient holding device where the container of the nutrients is waterproof, allowing preservation of the integrity of the active principle while eliminating direct contact with the skin, eliminating the possibilities of any dermatological or allergic reactions.

This container is then placed in various apparatus (i.e., hats, clothing, trochanter belts, lockets, pedal devices, etc.) to form the invention.

### SUMMARY OF THE INVENTION.

The invention relates to a nutrient-holding device which requires a compartment which houses nutrients. These active agents (nutrients, metabolites) have the ability to emit photons (electromagnetic waves) that can stimulate reflex zones located on the surface of the body (head, neck, back, foot, pelvis, umbilicus, chest, solar plexus, etc.). These agents deliver natural frequencies and radiations which affect reflex zones located throughout the body surfaces by placement of said device on the surface of various locations on the body. The subject is therefore able to wear these agents and be afforded the envisioned therapeutic effect continually.

According to the theory of the invention, the nutritional supplements used in this invention give off natural frequencies and radiations that affect reflex zones to correct posture and achieve a partial or complete restoration of the spinal physiological ailment and therefore prevent or cure disorders associated with spine disequilibrium such as musculoskeletal pain.

Those parts of the body which receive stimuli at the termination of peripheral afferent fibers are known as receptors. The human body is receptor-driven. Eighty percent of neurology is afferent, while only twenty percent efferent. Of this eighty percent, eighty percent are mechanoreceptors, and eighty percent of those are spinal proprioceptors (involved in posture and protecting the central nervous system).

The most abundant type of sensory endings in the body are non-encapsulated afferent endings found in practically all epithelia of the body, in connective tissue and in serous membranes. Therefore, although the feet are important sites of mechanoreception, they are by no means solely responsible for the enormous sensory (mechanoreceptive) proprioceptive input from the entire surface of the body.

This invention, therefore, uses the active agent nutrients encased in a device that holds it on the body at various locations to affect reflex zones by stimulating mechanoreceptors.

The stimulated mechanoreceptors cause a reflex to correct the posture by affecting the musculature to move the body back into alignment. They do this by making weak muscles stronger. In the presence of asymmetry, there are commonly associated

weak muscles. Their weakness is in part due to fatigue in trying to move the body back into symmetry.

Typical examples of appropriate active agents are vitamins, minerals (metabolites), RNA, DNA, fatty acids and other liquids, herbs, blue-green algae, homeopaths, hormones and enzymes.

The means of the invention is to place the nutritional supplements on the body utilizing a device which contains these active principles. The active principles stimulate mechano-receptors that fire afferent nerve impulses to the central nervous system. This is the first part of the reflex arc.

The central nervous system then sends back efferent impulses to the muscles that move bones back into symmetry. This is the function of the invention resulting in improved posture, as well as the second part of the reflex arc.

Although the exact mechanism involved in stimulation of the proprioceptive reflex is not well understood, it is believed that these agents (nutrients, nutritional supplements, metabolites) produce natural frequencies and radiations which affect the reflex zones. These agents may be selected from the following groups of metabolites which are listed as illustrative examples:

Herbs/phyto-nutrients: grape seed extract, garlic, flax seed oil, ginseng, ginger, echinacea, chlorella, bromelain, beetroot algae, bee pollen etc.

Amino Acids: L-acetyl-glucosamine, L-taurine, L-valine, inosine, GABA, creatine monohydrate, L-alanine, L-caratine etc.

Enzymes: amylase, betaine, lipase, pancreatin, pepsin, trypsin.

Vitamins: Vitamin A, Beta Carotene, Vitamin C, Vitamin D, Vitamin E, Niacin, Biotin, Inositol, PABA, Vitamin B12, Vitamin B6, Vitamin B5, Vitamin B1, Vitamin B2, Folic Acid.

Glandular and Protomophagens: DHEA, EPA, Coenzyme Q-10, Chrondrotin Sulfate, Chitin, Bovine Cartilage.

Hormones: DHEA, Melatonin, Pregnenolone, DMAE, CMO, NADH, HGH.

Minerals: Metabolites, consisting of organic sources of calcium, phosphorous, iron, potassium, iodine, bone meal, shark cartilage, etc.

The nutrient-holding device consists of a variety of housings that are waterproof and are specifically adapted to hold the nutrients adjacent to the body at various locations. The agent is separated from the body only by a thin layer of this waterproof housing. This waterproof container may be made of plastic, leather, any metal (except lead), wood, glass, fiberglass, nylon, etc.

Besides holding the agent next to the body, the waterproof holding device preserves the integrity of the active principle while eliminating direct contact with the skin and any possibility of dermatological or allergic reactions.

The therapeutic method (according to the invention) is that, by wearing the resulting nutrient-holding devices at various locations on the body, the expected therapeutic effect can be realized continually. The nutrients in the device exert a stimulatory action on the numerous proprioceptive nerve endings on the skin, thereby increasing muscle strength of weak muscles commonly associated with imperfect symmetry. This results in improved posture. Muscles are all attached to the body frame. It is their pull against these attachments that affect the positioning of the body (posture).

### DETAILED DESCRIPTION OF DRAWINGS

FIG. I-V Clinical Evaluation

FIG I. A P View of body

Head tilt is determined by observing the difference in height of the external auditory meatus. The clinician places his index fingers into the ear canal and observes any differences in height. The lower ear box is checked.

Shoulder balance is detected by palpitation of the shoulders, visual examination, and double checked by the following test: with both arms dangling straight down, the clinician brings both hands to the midline and observes the differences between both forefingers. The long arm indicates the low shoulder on that side. This lower box is checked.

FIG. II

Pelvic girdle rotation is detected by the following test: with the patient in the standing position, the patient raises both arms perpendicular to the body and parallel to the floor, the clinician joins both hands in the midline. The clinician than measures and observes the differences between both forefingers. The long arm indicates anterior rotation of the pectoral girdle. The appropriate box is checked indicating the long arm.

FIG. III

Now, the patient swings his arms directly over his head, the clinician than brings his hands to the midline and again measures the difference between both forefingers. The short arm indicates a contracted and weak psoas muscle on that side causing restriction in movement of the diaphragm on that side. This box is checked to mark the asymmetry.

FIG. IV

The low shoulder is observed and the appropriate box is marked. The high side of the pelvis is palpated and the appropriate box checked.

FIG. V

The patient is lying face up. The legs are tractioned and brought to the midline. The superior medial maleolas indicates the short leg side which usually corresponds to the high side of the pelvis.

The box is checked indicating the short leg side and the side of the superior medical maleous. (the medical maleous is indicated by arrows in the drawing).

FIG. VI-XV: The various types of nutrient holding devices that together with the active principle (nutrients, nutritional supplements, metabolites) make up the invention are illustrated for the purpose of illustration and without restriction.

FIG. VI A. Illustrates a hat, the nutrient holding device is incorporated in a band (1) that goes around the circumference of the hat holding the active principle next to the skull.

FIG. VI B. This longitudinal cross-section of the band shows the waterproof housing (2) of the band, which makes up the compartment which encloses the active principal.

FIG. VII A. Pictures the nutrient holding devices as a locket or disc. This AP view shows the outer plastic covering of the devices (3), a clasp (2), and chain (1), which allows the device to hang around the neck of the subject for contact at locations on the chest or solar plexus. The diameter of the locket is 3 inches, the width is 0.25 inches.

FIG. VII B. Pictures of a cross sectional view of VII A illustrating the plastic membrane around the device (4) and the resulting waterproof compartment (5) containing the active principle.

FIG. VIII. A. Illustrates an AP view of a pedal device, which may be the sole of a shoe, a pedal insert or the base of a sandal. The dotted outline indicates the position of the active principle, which is sandwiched between a fabric, leather material, or some other appropriate material (1) that pedal inserts could be made of.

FIG. VIII. B. Illustrates a longitudinal cross section of this pedal. The outer covering (2) of the device may be composed of leather, plastic foam or gel, or rubber surrounds a waterproof housing (3) which contains the active principle (4).

FIG. IX. A. Pictures the nutrient holding device as a belt. This AP view shows the outer covering (1) of the belt, which is preferable leather, but may be plastic or other material. The dotted line indicates the location of the active principle, which would be held next to the subject's waist in this particular device.

FIG. IX. B. Is a longitudinal cross section of IX A. illustrated the outer covering (1) that surrounds a waterproof compartment that may be in the form of a plastic insert (2) which surrounds the active principle (3).

FIG. X. A. Pictures an AP view of the nutrient holding device as veterinary apparel. In this case a horse blanket is illustrated. The dotted line indicates the area where the active principle is located between the outer fabric of the horse blanket and the under surface of the horse blanket.

FIG. X. B. This longitudinal cross sectional view of XA shows the outer fabric (2) of the device which surrounds a waterproof housing (3) which houses the active principle (4). This waterproof housing may be comprised of plastic gel or foam which may be compartmentalized to allow the active agent to cover the maximum surface area of the blanket.

FIG. XI. A. Depicts a drawing of beads or stainless steal ball which are 20× the actual size. The balls are hollow and constructed of waterproof materials that compose a holding device.

FIG. XI. B. Depicts a magnified cross section diagram illustrating the hard metal outer shell (1) and the compartment (2) which contains the active principle.

FIG. XI. C. When these balls are mixed with dental cement or amalgam, they can form a holding device that can be incorporated into a filling, thereby affording the envisioned therapeutic effect continually through this dental implant. Thus, an ordinary filling is transformed into a proprioceptive device.

FIG. XII. A. This is of a mattress (1) that has the active principle sandwiched between two layers of fabric.

FIG. XII. B. Pictures of a longitudinal cross-section of FIG. XII A, a waterproof compartment (3) lies under the fabric of the mattress (2). This compartment (3) contains the active principle (4), which can supply the envisioned therapeutic effect while resting.

FIG. XIII. A. depicts a spool of hollow thread. In the magnified view (1) of the thread showing that it is hollow (2). The active principle may be incorporated into such a thread. Then the resulting fiber becomes a nutrient holding device that can hold the active principle. The fiber must be waterproof to protect the integrity of the active agent. Then, the fiber can be woven into various articles of clothing that would provide the envisioned therapeutic effect while being worn.

FIG. XIII. B. Illustrates a cross-section of such a fiber, the waterproof housing (4), and the resulting compartment (5) for the active principle

FIG. XIII. C. Depicts various articles of clothing that could be considered holding devices when such fibers are utilized in producing clothing. Specifically, a shirt (6), gloves (7), pants (8), and socks (9) are illustrated.

FIG. XIV. A. Illustrates a watchband as a holding device for the active principle. The active principle is sandwiched between two pieces of leather.

FIG. XIV. B. Pictures a cross-section of this watchband, the leather material (1) surrounds a waterproof plastic insert (2) which contains the active principle (3).

FIG. XV. A. The AP view of a ring can act as a nutrient holding device. It is composed of metal, gold, silver, platinum, copper, etc.

FIG. XV. B. As seen in the cross sectional view, the hollow chamber of the ring can act as a nutrient holding device for the active principle thus providing the envisioned therapeutic response while being worn.

PREFERED EMBODIMENTS

| | |
|---|---|
| #1 | 3 tablespoons of a blue-green algae |
| #2 | 1 tablespoon of blue-green algae |
| | 1 tablespoon of Immuno-Faster (a mixture of glandulars from |
| | Douglas Laboratories) |
| | 1 tablespoon of Ultrapreventive III (a multi-vitamin-mineral formula |
| | from Douglas Laboratories) |
| #3 | 3 tablespoons of Ultrapreventive III powder (Douglas Laboratories) |
| #4 | 3 grams of 1000-k Alternative Formula (a prescription formula) |
| | 1 gram of DHEA |
| | 1 gram of Pregnenolone |
| | 50,000 units of Beta Carotene |
| | Ultra B-Complex tablets (4) tablets |
| | Natural C 500 (4) tablets |
| | 100 mg of Pyenogenol |
| | 1,200 units of Multi-E-400, Natural Dry |
| | 3 grams of Free Form Amino Caps |
| | 1 gram of Max-Taurine |
| | 1 gram of L-Lysine |
| | 1 gram of Creatine Monohydrate |
| | 2 grams of Ora-Multi (blend of 9 glandulars) (Douglas Labratories) |
| | 5 capsules of Herbplex (Douglas Laboratories) |
| #5 | 500 mg of Balanced-B complex |
| | 20 grams of Goldenseal Root Powder |
| | 300 mg of Ginkgo Biloba |
| | 1 gram of Echinacea |
| | 8 capsules of Herbplex |
| | Vitamin D Formula 3,000 IU |
| | Multi-E-400 (4) Capsules |
| | 3 grams of Chlorella Plus |
| | 6 capsules of Immuno-Forte |
| | (The above formulations are from Douglas Laboratories) |
| #6 | 2 teaspoons of Ultrapreventive III Powder (Douglas Laboratories) |
| | 1 teaspoon of Ultragreen Powder (Douglas Laboratories) |
| | 3 grams of Ori-Multi- a blend of glandular (Douglas Laboratories) |
| | 4 capsules of HerbPlex (Douglas Laboratories) |
| | 4 capsules of Aminoplus |
| | 1 teaspoon of Bee Pollen Granules |
| | 3 capsules of Ultramate Balance (prescription Formula 47) |
| | Shark Cartilage - 400 mg (Burn Stone Labs) |
| | Octacosonal - 1000 mcg (Burn Stone Labs) |
| | Chromium Pieolonate 200 mcg (Burn Stone Labs) |

The instant invention will become more understood by referring to the following examples, which are given to picture more readily the invention rather than limit its scope.

### EXAMPLE I

Twenty patients were examined and found to have a torsion lesion of the SI joint using SOT analysis. Classically, there are certain indicators that appear in this categorization specifically a short leg on the high side of the SI joint. Also associated with this lesion are muscle weaknesses involving the anterior leg flexors, the tensor facia lata and the psoas. Postural distortions common with this group of patients include pelvic tilt, shoulder height imbalance, pectoral girdle rotation, head tilt and short leg.

These selected patients exhibiting this category of parameters were subjected to a clinical postural examination as illustrated in FIG. I-FIG. V.

A baseline of postural distortion was thus established. The results were recorded.

Blindness of the study was assured by the following procedure.

Two color-coded lockets as depicted in FIG. VII A. were created. The red locket contained the active principle in the waterproof chamber (5) as illustrated in VII B. The gold locket contained sugar in the waterproof container (5) as depicted in VII B. Thus an active locket and placebo locket of the same shape, size and weight were created. Each disc proper was put into an identically colored small sack. The chain was allowed to extend out of the sack, which was closed by a small rubber band. This would allow for easy hanging of the device around the patient's neck allowing the device to be placed on the subject's chest for subsequent evaluation. Both discs were than placed into a black bag for random drawing.

The subject would than draw a disc that had a colored sac on it with the chain extending from it from the black bag.

He would then place the chain around his neck allowing the disc in the colored sac to lie on his chest. Another evaluation was done on the subject using the criteria illustrated in FIG. I to FIG. V. The results were recorded. The disc was then removed from the sac and the color recorded. The disc the colored sac would then be returned to the black bag for subsequent random drawing. The remainder of the twenty subjects were likewise examined. In this way a separate placebo control group of 8 individuals was generated, along with a group of 12, which had the intervention of the active principle.

Results: All subjects with active principle intervention showed improved posture with equalization of the vast majority of the seven parameters measured. The control group on the other hand did not show any improvement in the parameters measured, maintaining the original distortion observed in the baseline observations.

Conclusion: (by screening potential subjects for the existence of the sacroiliac torsion lesion) over 90% of the subjects showed improvement of the active principle as compared to the control group which did not show any significant improvement.

Note: Several subjects were permitted to wear the active principle locket home. When in 3 days they returned, this torsion lesion was examined. None of the indicators of this lesion could be found. However, when the locket was removed classical SI torsion indicators were found, including short leg, muscle weakness, and postural distortions.

Note 2: The above experiment demonstrates the efficacy of the preferred embodiment being:

1 tablespoon of blue green algae

1 tablespoon of ultrapreventitive III Powder (Douglas Laboratories)

1 tablespoon of Immune-Forte (a mixture of glandulars from Douglas Laboratories)

Therefore, in this instant case, in order to make the invention this above preferred embodiment is encased in a flat plastic enclosure of three inches. The resulting locket is hung on a chain. (FIG. VII. A.)

To use, simply place this locket around the neck allowing the active principle to lie on the chest of the subject. The locket is worn to provide the continued envisioned therapeutic effect of posture correction.

### EXAMPLE II

This relationship between muscle tone and posture is again utilized in another double-blind study that is detailed in the following experiment by Dominic Frio, D. C. in Hoboken, N.J.

This experiment is an example of how the device can be used at multiple locations with success in making weak muscles stronger. The following locations are used: the umbilicus, the left and right upper back, the lower back (L-5) and the solar plexus.

The preferred embodiments being exemplified here are three tablespoons of blue-green algae powder uncased in a plastic coated disc three inches in diameter.

Dr. Dominic Frio is a chiropractor that has practiced in New Jersey for 25 years and a U.S. citizen for 10 years.

He was given two discs to evaluate using manual muscle testing. The discs were all of the same size, shape, and weight. They were color-coded gold and red. He was not told which was the placebo and which was the active agent. An assistant put each disc into separate envelopes.

His evaluation consisted of locating a weak muscle, which was recorded (see accompanying chart). His assistant presented the two identical envelopes to him from outside the examination room. Then he would ask his patient to pick one of the envelopes. He would then place to envelope on the body at various locations, which were noted. He than retested the muscle to determine if it had weakened or strengthened. This data was than recorded.

The color of the disc was only logged after taking it out of the envelope. The disc was given back to his assistant, who put it in the envelope for the next trial. Forty-four patients were used in this study.

Thus a control group consisting of 16 subjects was generated and provided these results:

10 muscles remained weak

6 muscles tested stronger

A Separate group that used the intervention of the nutritional supplement was generated and produced these results:

2 muscles remained weak

26 tested stronger

Therefore, blue-green algae powder may be considered an active principle since it has demonstrated its ability to make a muscle stronger that previously tested weak, thus it effects muscle tone and the posture.

### EXAMPLE III

The body is in a constant state of contracture, which holds the body's posture and maintains firmness called muscle tone. Any weakness of opposing muscles or antagonistic muscles will result in the skeleton having to move out of its normal position. Therefore, balancing this muscle tone results in improved posture

Keeping this relationship in mind, please examine the results of a placebo controlled; double blind study done by an independent investigator. This pilot study was conducted by Renee Garcie, D. C. in Shreveport, La.

This is an example of placement of the active principle on the umbilicus. The preferred embodiments being exemplified are three tablespoons of Ultra Preventative III encased in the buckle of a support belt that was sent to her with an identical placebo (see appendix for ingredients of Ultra Preventive II Powder).

The active principle was hidden in a belt buckle and marked “A” on the underside. The mark was covered by tape. The other belt likewise contained sugar and was marked “PL” on the underside of the belt buckle, and was covered by identical tape.

The first subject was analyzed for strength of the psoas muscles. A leg movement was conducted. The results were logged.

Next, the subject drew out one of the identical belts out of a black bag. The subject put the belt on. The psoas muscles were tested again. The leg lengths were also noted. Any change in strength of the psoas and leg were also noted. The subject then put the remaining belt on. The psoas muscles were tested and leg lengths were measured. The results were noted.

The tape was then removed from the second belt, and the belt was identified and logged. The other belt's identity was also logged.

Seven patients were put through the same regimen, generating a placebo group and an identical group testing the active agent.

Although the subjects were used as their own controls, the method of randomly drawing the belts from a black bag and revealing the identity of the belts after testing parameters preserves the integrity of this randomized double blind study.

The results were as follows:

TEST RESULTS

| | | | Intervention of Active |
|---|---|---|---|
| No. | Preliminary Findings | Control | Agent |
| 1. | 21 year-old W/F | | |
| | Short left leg | No change | Equalized leg length |
| | Weak left psoas | No change | Strong left psoas |
| 2. | 20 year-old W/F | | |
| | Even leg lengths | — | — |
| | Weak right psoas | No change | Strong left psoas |
| 3. | 52 year old W/F | | |
| | Short right leg | No change | No change |
| | Weak left psoas | No change | No change |
| 4. | 43 year-old W/F | | |
| | Short right leg | No change | Equalized leg length |
| | Weak right psoas | No change | No change |
| 5. | 57 year-old W/F | | |
| | Short right leg | No change | Equalized leg length |
| | Weak right psoas | No change | No change |
| 6. | 49 year-old W/M | | |
| | Even leg lengths | — | — |
| | Weak right psoas | No change | No change |
| 7. | 43 year-old W/M | | |
| | Short right leg | No change | Equalized leg length |
| | Weak right psoas | No change | Strong right psoas |

### Results

No change in leg length and psoas muscle strength was noted in the contact group.

The active agent intervention showed two patients with no change in leg length or psoas strength.

Four subjects, each of whom had short leg in preliminary measurements, had equal leg lengths with the intervention of the active agent.

Note: Two subjects had even leg lengths in the preliminary measurement, therefore a (--) in the chart indicates No comparison was made.

Four of the seven subjects showed an increase in psoas strength, while three subjects' psoas strength measurements remained weak.

### Discussion

The control group did not show any change in either leg length or psoas muscle strength.

The group with the active agent showed changes in leg length and/or muscle strength in four patients.

This change can only be due to the intervention of the active agent.

The significance of an increased in muscle strength is obvious.

The significance of leg length equalization can only be due to an increase in muscle tone resulting in movement of the pelvis, thus equalizing leg length.

### EXAMPLE IV

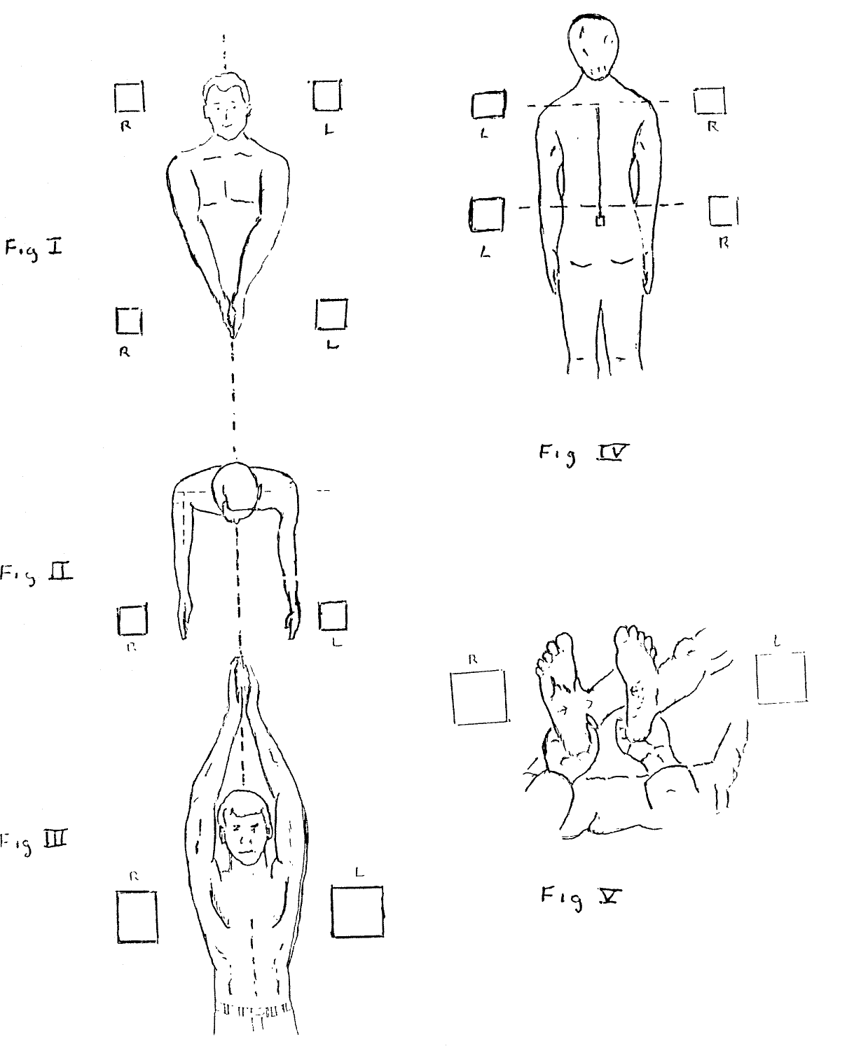

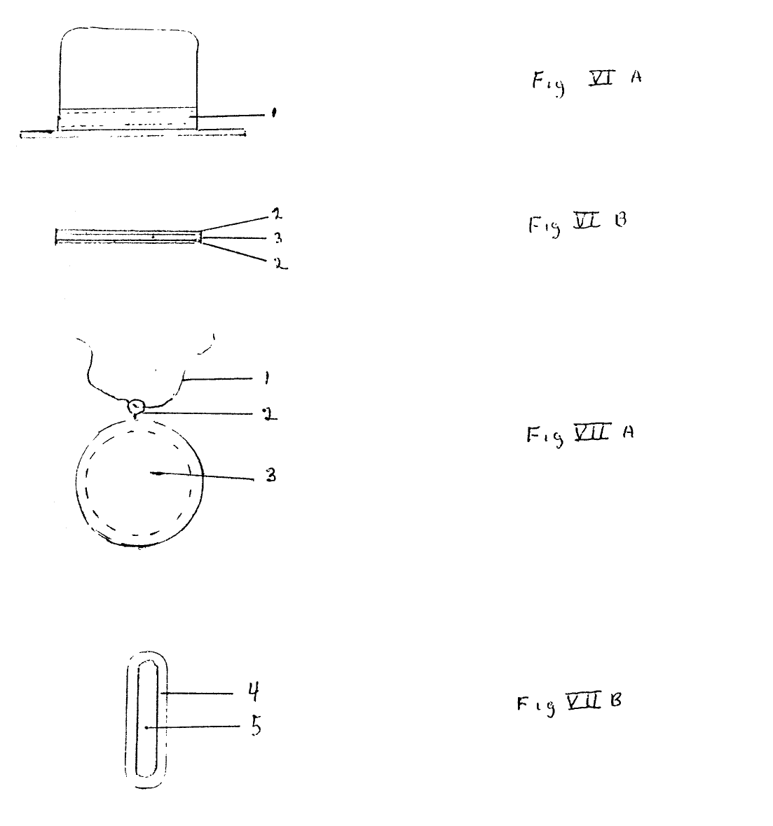

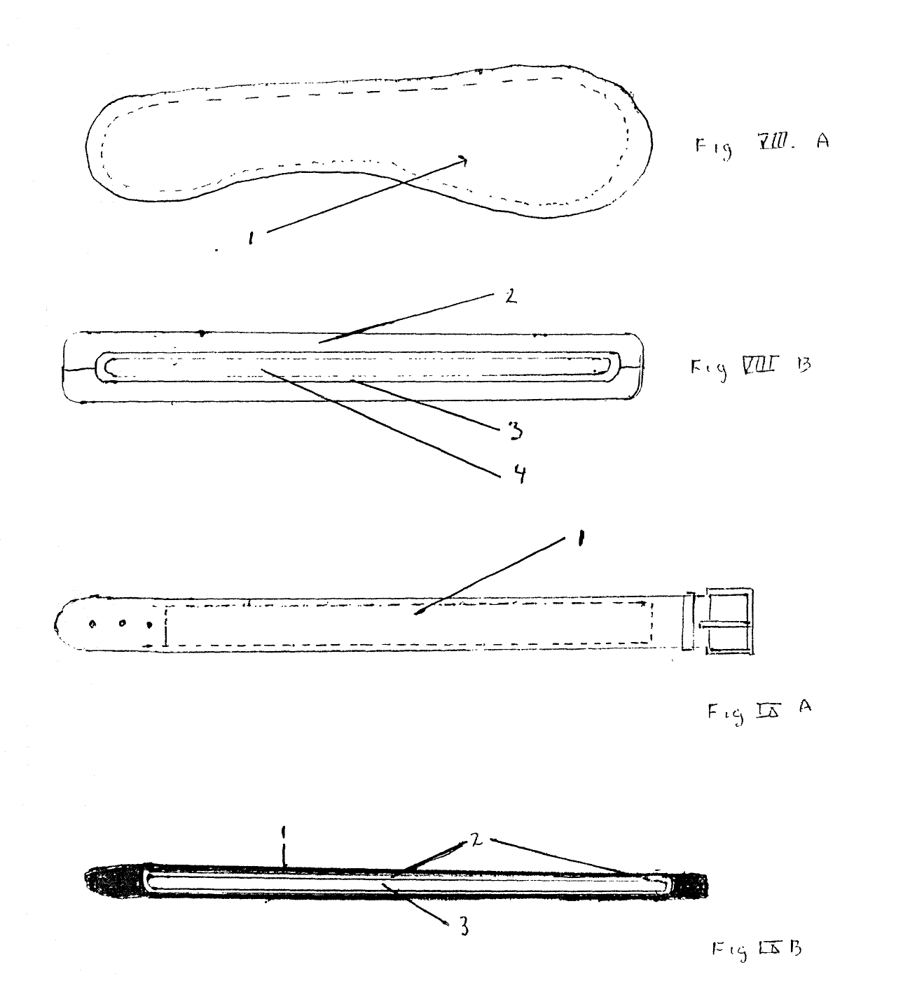

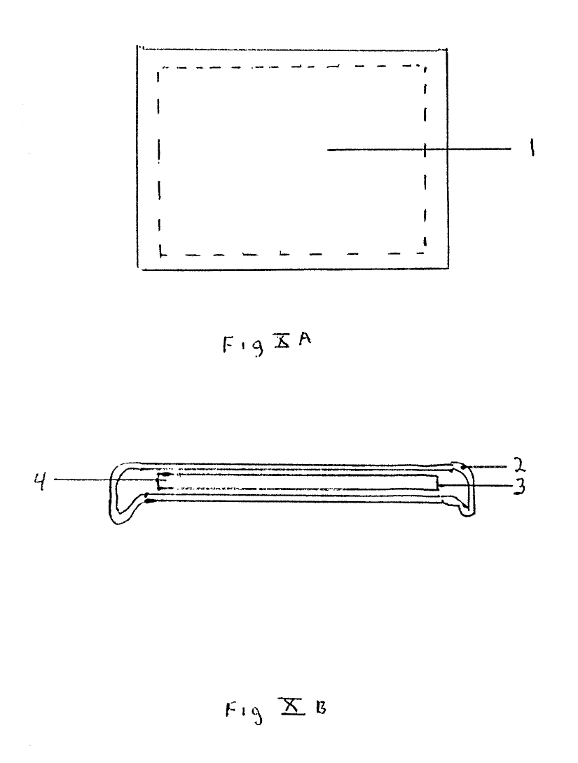

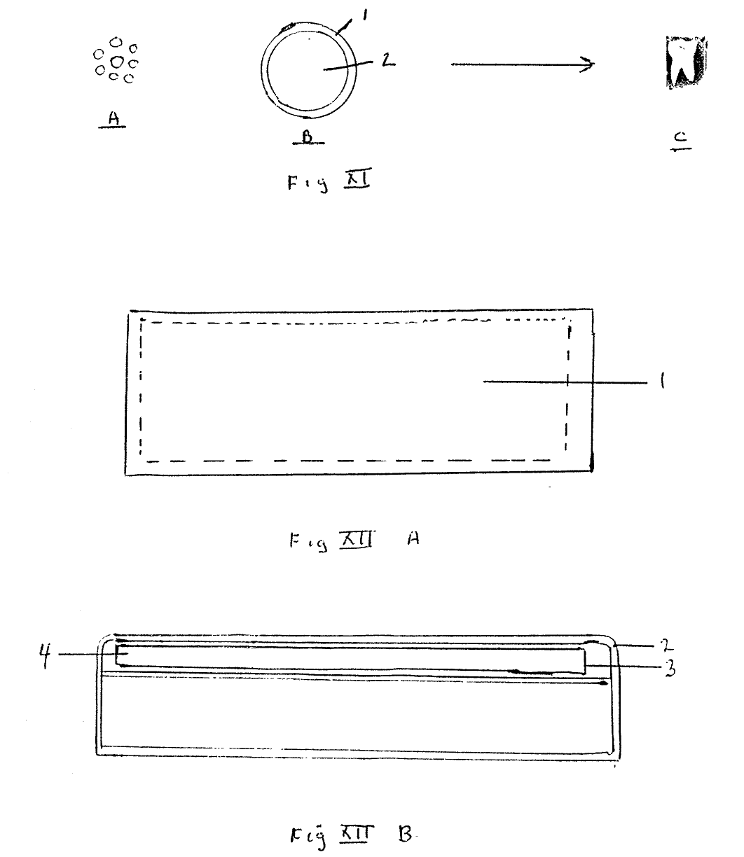

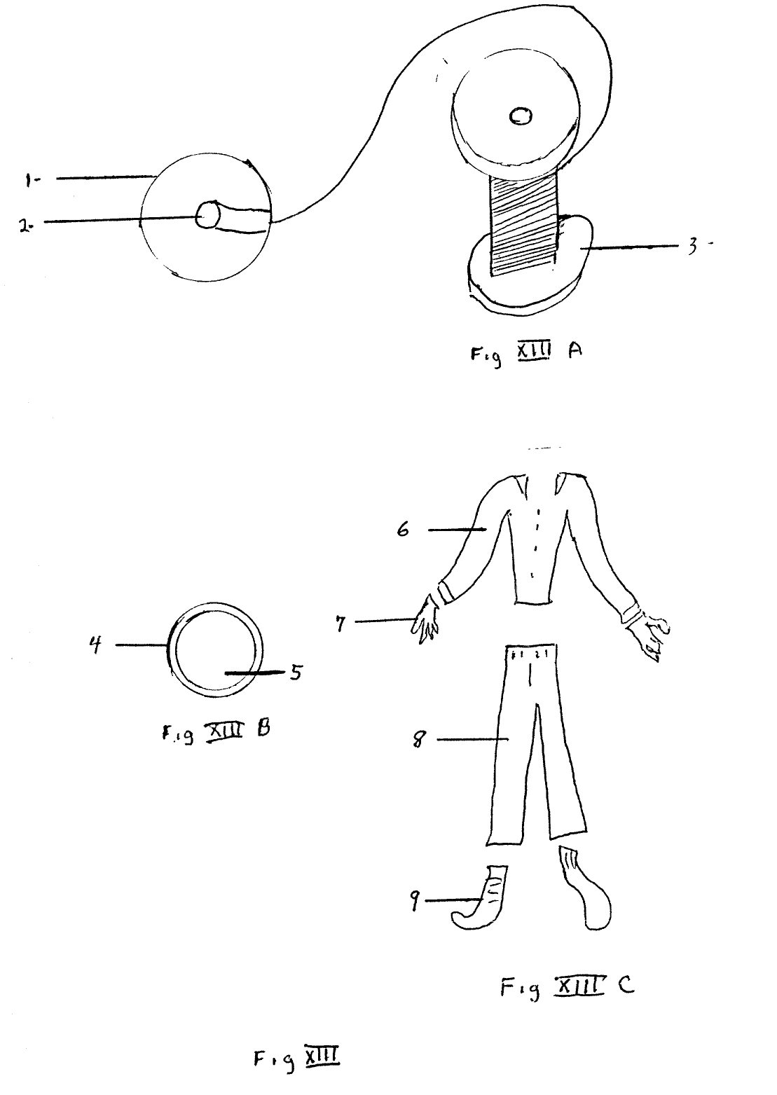

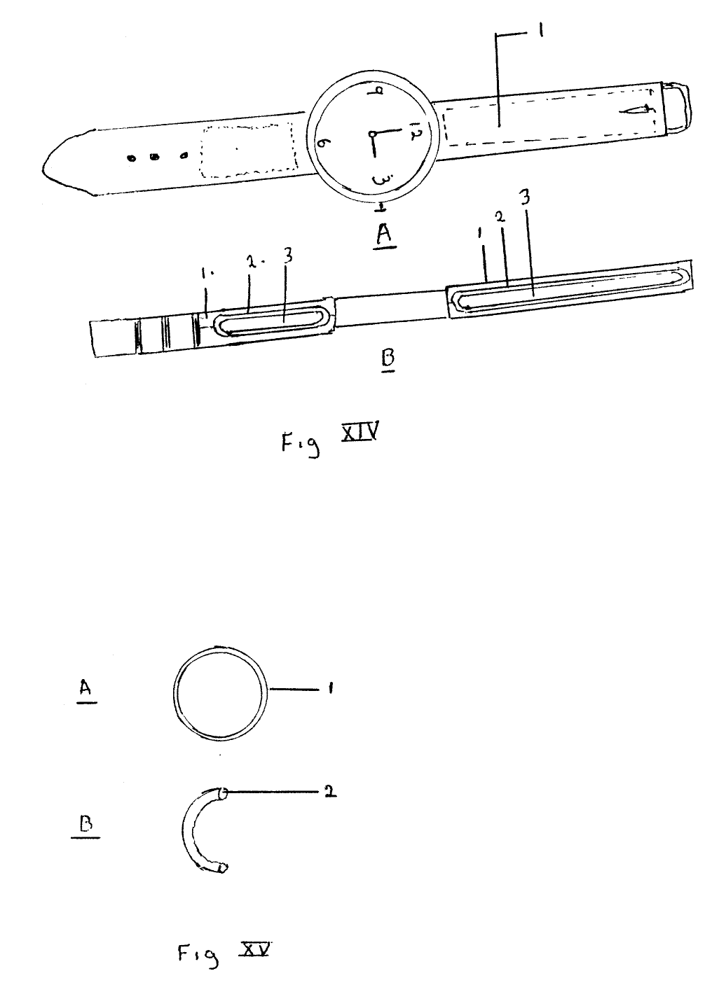

The ability of this invention to correct posture when used in a hat (FIG. 1) was confirmed by repeated observations of over 50 separate and distinct instances. Baseline observations were conducted of head tilt, shoulder height, anterior rotation of the pectoral girdle, superior ilium and short leg. When the hat was put on the response to the resulting placement of the active principle around the circumference of the skull was immediately observed as an improvement in posture using the above five criteria.

The preferred embodiment utilized in the above hatband was preformed embodiment #4.

### EXAMPLE V

The preferred embodiment #5 was utilized in the pedal device VIII A. In this case the pedal device was fashioned into a pair of sandals. The active principle (4) was encased in the waterproof housing. FIG. VIII B (3). Baseline Postural Analysis (FIG. I-V) was done on 20 subjects barefooted. The results were observed. The subjects would then slip into the above sandals. The observed postural improvement was immediate as the subjects reported the postural clinical analysis.

### EXAMPLE VI

The ingredients in preferred embodiment #6 were quadrupled and utilized as the active principle in a horse blanket (FIG. X A). In FIG. X B. the active principle (4) was placed in the waterproof housing (3) between the outer covering (2) of the blanket. Such a blanket was sent to Kathy KcKinley Johnson of Oakland Calif., a horse owner, for evaluation. She reported back that the horse she used the blanket on was less clumsy, and more controllable with the blanket on. Since function precedes structure, this observation could be attributed to increased symmetry resulting in better performance.

### EXAMPLE VII

In FIG. XI B, the active principle (2) is surrounded by waterproof metal or ceramic material forming small balls which may be incorporated with amalgam to fill cavities, thus forming a permanent proprioceptive implant.

To observe this potential first hand, I performed baseline clinical analysis (FIG. I-V) on 10 subject, observed their distortions and watched them disappear when placing a tablet of “Centrum” or some other multi-vitamin between their molars. The examiner is invited to experience this potential first hand.

### EXAMPLE VIII

To examine how besides moving the bones into symmetry, use of this device results in increased strength and endurance, Steven J. Cutney, the strength and conditioning coach of Mt. Vernon High School performed the following placebo controlled double blind study.

He was given two identical medallions. They had the same size, shape, weight, and color. He was not told which was the placebo and which was the active agent. The medallions were coded for differentiation.

Twenty subjects were tested on a bench press using four separate lifts. Prior to testing, all athletes were provided with a warm-up and stretching session of uniform intensity and duration. The time between each lift was about five minutes.

All subjects lifted 90 percent of their IRM (one repetition maximum). The first lift is done to establish a baseline and is done without having a medallion on the body, The objective results are recorded as whole numbers, indicating the number if repetitions completed through a full range of motion. The subject then draws a medallion that has been encased in an envelope out of a black bag. The bag had both medallions encased in separate envelopes.

Before starting the second lift, he places the envelope on the solar plexus of the subject. The instructions were repeated to the repeated to the subject, and he completed his maximum effort. The results were then recorded (see accompanying chart).

The third lift was done to establish a second baseline and is done without having a medallion on the body, the instructions were repeated and the subject completed his maximum effort. The results were then recorded.

The fourth lift was done with the same envelope on the subject's solar plexus. The instructions were repeated and the subject completed his maximum effort. The results were then recorded.

Upon completion of the lifts, the envelope was opened and the code was recorded. The medallion was put into a new envelope and returned to the black bag for subsequent tests.

The data was given to this investor for interpretation (see accompanying chart and statistical analysis in appendix).

### Conclusion

The subjects were able to perform their prescribed exercises with increased ability due to the intervention of the active principle (Agent A) as opposed to placebo intervention B.

A statistical analysis of the data is enclosed in the appendix.

### Discussion

Since structure precedes function this increased ability may be due to more functional joint angles due to increased symmetry or to an overall increased muscle tone required to produce this symmetry.

[image]

[image]

[image]

[image]

[image]

[image]

[image]

[image]

[image]

[image]

[image]

1- A nutrient holding device for treating and/or preventing physical problems associated with postural disequilibrium which consists of an article adapted to contact the body, said article incorporating a waterproof enclosure containing at least one nutritional supplement, whereby this nutrient material is only separated from the surface of the body by the thin covering of said nutrient holding device:

2- A nutrient holding device according to claim 1 wherein said device is a locket worn around the neck to lie on the chest or solar plexus.

3- A nutrient holding device according to claim 1 wherein said device is a band in the circumference of a hat.

4- A nutrient holding device according to claim 1 where said device is a pedal device which may be an inner sole consisting of two semi-rigid fabric layers bonded together around a waterproof housing containing the active principle (nutrients, metabolites).

5- A nutrient holding device according to claim 1 where in said device is a belt, where the nutrients are held in a waterproof plastic insert between two pieces of leather. Thereby, the active principle is held next to the subject's waist whose posture is improved while wearing said device.

6- A nutrient holding device according to claim 1 where the device is an article of jewelry, which is, hollow and contains an active principle.

7- A nutrient holding device according to claim where the active principle is put into a hollow fiber and woven into clothing thereby affording the envisioned therapeutic effect while wearing said clothing.

8- A nutrient holding device according to claim 1, where in said nutrient is in the form of a solid, powder, gel, or liquid.

9- A nutrient holding device according to claim 2 where said device is incorporated into furniture (i.e. mattresses and chairs) so that the envisioned therapeutic effect can be provided while resting.

10- A nutrient holding device according to claim 2 where the active principle is three tablespoons of blue-green algae powder.

11- A nutrient holding device according to claim (1) where the active principle if 1 tablespoon of Ultra-Preventive III, 1 tablespoon of Immuno-forte, and 1 tablespoon of blue-green algae.

12- A nutrient holding device according to claim 2 where the active principle consists of 3 tablespoons of Ultra-Preventive III Powder.

13- A nutrient holding device according to claim 1 where in said nutrients are selected from vitamins, minerals, blue-green algae, protomorphagens, glandulars, amino acids, herbs, enzymes, and derivations thereof.

14- A treatment or preventive method according to claim 1 for the physical troubles associated with structural disequilibrium which comprises placing nutrient holding device on the person in need of correction, the active principle will then stimulate propricceptive reflex zones located at the proximity of placement of said devices, resulting in increased muscle tone and corrected posture. Thus, by wearing said device, the envisioned therapeutic effect can be afforded continually

15- The method according to claim 14 where placement of the device occurs at the circumference of the head, the chest, the solar plexus, the umbilicus, the right and left shoulder, the lower back and the sole of the feet.

16- The method according to claim 1 where placement of the device is at the proximity of clothing that the active principle has been incorporated into the weave thereof.

17- A nutrient holding device in the form of vetenary apparel according to claim 1 where the envisioned therapeutic effect is afforded to animals by having the animals wear the active principal next to their epidermis. (i.e. horse blanket)

18- A method of improving muscle strength and endurance while conducting physical activities (exercise) by wearing the nutrient holding device during such activities.

19- A nutrient holding device that is a hollow metal or ceramic is a hollow metal or ceramic ball or chamber that is waterproof and being of a small diameter that it can be mixed with dental cement or amalgam to be used to fill a cavity, thus becoming a proprioceptive stimulating implant by stimulating the rich proprioceptive nerve endings of the jaw.
